(19) United States
(12) Patent Application Publication
(10) Pub. No.: US 2026/0263108 A1
(43) Pub. Date: Sep. 10, 2026

(54) THROMBUS REMOVAL SYSTEMS AND ASSOCIATED METHODS

(71) Applicant: Shifamed Holdings, LLC, Campbell, CA (US)

(72) Inventors: Praveen Krishna DALA, San Ramon, CA (US); Aadel AL-JADDA, San Carlos, CA (US); Matthew T. MUNOZ, Santa Cruz, CA (US); Tom SAUL, Portland, OR (US); Christopher Scott JONES, Menlo Park, CA (US)

(21) Appl. No.: 18/994,012

(22) PCT Filed: Jul. 14, 2023
(86) PCT No.: PCT/US2023/070286

§ 371 Date: Jan. 13, 2025

Related U.S. Application Data
(60) Provisional application No. 63/368,444, filed on Jul. 14, 2022.
(60) Provisional application No. 63/373,386, filed on Aug. 24, 2022.

(51) Int. Cl. A61B 17/3203; A61B 17/00; A61M 25/00

(52) U.S. Cl. CPC A61B 17/32037; A61M 25/0021; A61B 2017/00022; A61B 2017/00154; A61B 2217/005; A61M 2025/0003

(57) ABSTRACT

The present technology relates to systems and methods for removing a thrombus from a blood vessel of a patient. In some embodiments, the present technology is directed to systems including an elongated catheter having a distal portion configured to be positioned within the blood vessel of the patient, a proximal portion configured to be external to the patient, and a lumen extending therebetween. The system can also include a fluid delivery mechanism coupled with a fluid lumen and configured to apply fluid to at least partially fragment the thrombus, and an aspiration mechanism fluidly coupled to an aspiration lumen and configured to aspirate the fragmented thrombus.

### CLAIM OF PRIORITY

This application claims the benefit of U.S. Provisional Application Nos. 63/368,444, filed Jul. 14, 2022, and 63/373,386, filed Aug. 24, 2022, the entirety of which are herein incorporated by reference in their entirety for all purposes.

### RELATED APPLICATIONS

This application is related to International Application Nos. PCT/US2021/020915, and PCT/US2022/033024, the disclosures of which are incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present technology generally relates to medical devices and, in particular, to systems and methods for removing thrombus, for example from the veinous system, using mechanical thrombectomy.

### BACKGROUND

Thrombotic material may lead to a blockage in blood flow within the vasculature of a mammal. Such blockages may occur in varied regions within the body, such as within the pulmonary system, peripheral vasculature, deep vasculature, or brain. Pulmonary embolisms typically arise when a thrombus originating from another part of the body (e.g., a vein in the pelvis or leg) becomes dislodged and travels to the lungs. Anticoagulation therapy is the current standard of care for treating pulmonary embolisms, but may not be effective in some patients.

Conventional mechanical thrombectomy devices for treating venous thromboembolism (VTE) utilize aspiration (suction) and/or rotating macerators. Such conventional devices may not be capable of navigating the tortuous vascular anatomy and/or effectively removing thrombotic material. Devices designed for treating deep vein thrombus (DVT) require navigating tortuous paths and small vessels which in turn do not allow for large aspiration lumens and rotating or moving physical mechanisms. Due to these and other limitations most DVT patients are left untreated as long as the risk of limb ischemia is low. In more urgent cases, they are treated with catheter-directed thrombolysis or lytic therapy to break up a clot over the course of many hours or days. In clinical use conventional DVT devices alleviate the clot burden temporarily but do not effectively treat the patient such that patients often return later with re-emergence of DVT.

Devices for treating pulmonary embolisms (PE) typically have relatively larger profiles which allow for more aspiration or larger aspiration catheter bore to better remove organized, hard clot but suffer from an inability to navigate beyond the truncus.

More recently treatment of VTE has trended towards aspiration-based catheters; however, these devices require a tradeoff between effectiveness (more aspiration) and blood loss. More aspiration causes more blood to be removed, which puts the patient at risk. As a result, clinicians stop procedures after a set amount of time or blood removal even if they have more clot they would otherwise choose to remove.

Systems developed to address the tradeoff between aspiration and blood loss have been largely unsuccessful. One approach involves saving the blood by recirculating cleaned blood back into the patient. This limits blood loss but adds many clinical and technical complications. Another approach in current use involves automated and physician-guided controls of the aspiration mechanism to reduce its use. Such systems detect when a clot is engaged in the catheter tip and cycle down or completely turn off aspiration and allow for the vacuum in the catheter to pull in the material. In use these systems have demonstrated several problems. First, it takes a long time to detect clogging with occlusive material (e.g. engagement with the clot).

Such systems detect the “clogged state” based on pressures in the catheter, for example, full vacuum is reached in the catheter or a pressure differential between vacuum and ambient pressure approaches reaches a maximum. Due to compliance in the catheter system, fluid turbulence, and other factors, it can take many seconds before the “clogged state” parameters are settled. The system then may need to wait several more seconds to confirm the state. In practice, such systems can take about 20 seconds to detect occlusive material that has been caught in the catheter which defeats the purpose of limiting the aspiration time of use. Second, such systems often fail to detect a clogged state. Pressure in the catheter involves significant mechanisms (such as noise and or ringing) making it difficult to identify a clogged state based on absolute pressure. Third, even if the system successfully detects occlusive material has been captured, conventional systems lack the ability to remove hard clot through the catheter without high vacuum pressure, or can't remove the clot at all (in which case the practitioner may try to remove the clot by holding the clot at the distal end of the catheter via the vacuum and slowly removing the catheter with clot attached, often referred to as “lollipopping” the clot).

There remains the need for devices and methods to address these and other problems with existing thrombectomy systems. There remains the need for a fast, easy-to-use, and effective device for removing a variety of clot morphologies with limited blood loss.

### SUMMARY

A system for removing thrombus is provided, the system comprising: an elongated catheter having at least one aspiration lumen configured to remove thrombus material; an aspiration mechanism fluidly coupled to the aspiration lumen and configured to reduce pressure in the aspiration lumen; a pressure sensor configured to monitor a pressure inside the aspiration lumen; a valve disposed between the pressure sensor and the aspiration mechanism; and an electronic controller operatively coupled to the pressure sensor and the valve, the electronic controller being configured to open and close the valve and monitor the pressure within the aspiration lumen to determine if a clot is engaged with the elongated catheter or if the clot has been at least partially removed.

In one aspect, the electronic controller is configured to close the valve every 3-5 seconds of therapy.

In one aspect, a cycle time to open or close the valve is approximately 300 ms or less.

In another aspect, the electronic controller is configured to determine that a clot is engaged with the elongated catheter when a monitored pressure does not substantially increase after closing the valve.

In some aspects, the electronic controller is configured to determine that a clot has been at least partially removed when a monitored pressure increases after closing the valve.

In one aspect, the system is configured to remove no more than 45 ml of blood from the patient before detecting that the clot has been at least partially removed.

In some aspects, the valve has a valve state that comprises an open state, a closed state, an opening state, or a closing state.

In some aspects, the electronic controller is configured to determine that a clot is engaged if the valve moves from a closed state to an open state and the pressure within the aspiration lumen fails to exceed a clot engagement threshold.

In one aspect, the electronic controller is configured to determine that a clot is engaged if the valve moves from a closed state to an open state and a slope of the pressure within the aspiration lumen fails to exceed a clot engagement threshold.

In some aspects, the electronic controller is configured to assess a change in pressure within the aspiration lumen over time to determine if clot is engaged with the elongated catheter or if the clot has been at least partially removed.

In one aspect, the change in pressure within the aspiration lumen over time is an input into a correlation function against a pre-defined clot detection profile.

In some aspects, the change in pressure within the aspiration lumen over time are filtered and normalized.

In some aspects, the electronic controller is configured to provide the pressure within the aspiration lumen and the valve state to a trained machine learning model to determine if clot is engaged with the elongated catheter or if the clot has been at least partially removed.

A system for removing thrombus is provided, the system comprising: an elongated catheter having at least one aspiration lumen configured to remove thrombus material; an aspiration mechanism fluidly coupled to the aspiration lumen and configured to reduce pressure in the aspiration lumen; a valve coupled to the aspiration mechanism, the valve having a valve state that comprises an open state or a closed state; a first pressure sensor disposed distal to the valve and configured to monitor a first pressure inside the aspiration lumen; a second pressure sensor disposed proximal to the valve and configured to monitor a second pressure inside the aspiration lumen; and an electronic controller operatively coupled to the pressure sensor and the valve, the electronic controller being configured to open and close the valve and monitor the first pressure and second pressure within the aspiration lumen to determine if a clot is engaged with the elongated catheter or if the clot has been at least partially removed.

In one aspect, the electronic controller is configured to close the valve at most every 3-5 seconds of therapy.

In another aspect, a cycle time to open or close the valve is approximately 300 ms or less.

In some aspects, the electronic controller is configured to determine that a clot is engaged with the elongated catheter when a monitored pressure does not substantially increase after closing the valve.

In one aspect, the electronic controller is configured to determine that the clot has been at least partially removed from the elongated catheter when a monitored pressure increases after closing the valve.

In some aspects, the system is configured to remove no more than 45 ml of blood from the patient from at least partially removing the clot to detecting that the clot has been at least partially removed.

In one aspect, the electronic controller is configured to determine that a clot is engaged if the valve moves from a closed state to an open state and the first and second pressure within the aspiration lumen fails to exceed a clot engagement threshold.

In another aspect, the electronic controller is configured to determine that a clot is engaged if the valve moves from a closed state to an open state and a slope of the first and second pressure within the aspiration lumen fails to exceed a clot engagement threshold.

In some aspects, the electronic controller is configured to assess a change in pressures within the aspiration lumen over time to determine if clot is engaged with the elongated catheter or if the clot has been at least partially removed.

In one aspect, the change in pressures within the aspiration lumen over time is an input into a correlation function against a pre-defined clot detection profile.

In another aspect, the change in pressures within the aspiration lumen over time are filtered and normalized.

In some aspects, the electronic controller is configured to provide the first and second pressures within the aspiration lumen and the valve state to a trained machine learning model to determine if clot is engaged with the elongated catheter or if the clot has been at least partially removed.

A non-transitory computing device readable medium is provided having instructions stored thereon for determining a clot engagement state of a thrombectomy device, wherein the instructions are executable by a processor to cause a computing device to: receive a valve state of a thrombectomy device that indicates an open state if a distal end of the thrombectomy device is in fluid communication with an aspiration source or a closed state if the distal end is not in fluid communication with the aspiration source; receive one or more pressure measurements within the aspiration lumen; and evaluate the one or more pressure measurements and the valve state to determine the clot engagement state with the distal end of the thrombectomy device.

In some aspects, the instructions cause the computing device to close the valve at least every 3-5 seconds of therapy.

In one aspect, a cycle time to open or close the valve is approximately 300 ms or less.

In some aspects, the engagement state of the clot is determined to be engaged with the distal end of the thrombectomy device when the pressure measurements fail to increase after the valve state changes from the open state to the closed state.

In another aspect, the engagement state of the clot is determined to be at least partially removed from the distal end of the thrombectomy device when the pressure measurements increase after the valve state changes from the open state to the closed state.

In some aspects, the computing device is configured to detect that the engagement state of the clot before the thrombectomy device removes more than 45 ml of blood from the patient.

In another aspect, the computing device is configured to determine that the clot engagement state as engaged if the valve state moves from the closed state to the open state and the one or more pressures within the aspiration lumen fail to exceed a clot engagement threshold.

In one aspect, the computing device is configured to determine that the clot engagement state as engaged if the valve state moves from the closed state to the open state and a slope of the first and second pressure measurements from within the aspiration lumen fails to exceed a clot engagement threshold.

In another aspect, the computing device is configured to assess a change in pressures measurements within the aspiration lumen over time to determine the clot engagement state as clot engaged with the elongated catheter or as clot at least partially removed from the elongated catheter.

In one aspect, the change in pressures measurements within the aspiration lumen over time is an input into a correlation function against a pre-defined clot detection profile.

In another aspect, the change in pressures measurements within the aspiration lumen over time are filtered and normalized.

In another aspect, the computing device is configured to provide the one or more pressures measurements within the aspiration lumen and the valve state as an input to a trained machine learning model to determine the clot engagement state with the distal end of the thrombectomy device.

A non-transitory computing device readable medium is provided having instructions stored thereon for determining a clot engagement state of a thrombectomy device, wherein the instructions are executable by a processor to cause a computing device to: receive a valve state of a thrombectomy device that indicates valve open if a distal end of the thrombectomy device is in fluid communication with an aspiration source or valve closed if the distal end is not in fluid communication with the aspiration source; receive one or more pressure measurements within the aspiration lumen; and provide the valve state and the one or more pressures as inputs into a trained machine learning model; output the clot engagement state with the machine learning model.

In one aspect, the output comprises a probability of the clot engagement state.

In another aspect, the output comprises a binary clot engagement state.

In one aspect, the output comprises a discrete clot engagement state.

In another aspect, the discrete clot engagement state indicates if no clot is engaged, if clot is engaged, or if clot has been removed.

In some aspects, the output is indicated using audio, visual, and/or tactile feedback.

In one aspect, the computing device is configured to disable the aspiration source if the clot engagement state indicates that clot has been removed.

In another aspect, the valve state further indicates valve opening if the distal end of the thrombectomy device is transitioning from being not in fluid communication with the aspiration source to being in fluid communication with the aspiration source.

In another aspect, the valve state further indicates valve closing if the distal end of the thrombectomy device is transitioning from being in fluid communication with the aspiration source to being not in fluid communication with the aspiration source.

A method for removing thrombus is provided, comprising: introducing a distal portion of an elongated catheter into the body of a patient, the catheter including an aspiration lumen in fluid communication with the distal end for removal of thrombus; positioning a distal end of the catheter in the region of a target thrombus; reducing pressure in the aspiration lumen to generate a vacuum at the distal end of the catheter; closing or opening a valve in the aspiration lumen; monitoring pressure at least in the aspiration lumen; and identifying engagement of the target thrombus with the distal end based on the monitored pressure and a valve state of the valve.

A method for removing thrombus is provided, comprising: introducing a distal portion of an elongated catheter into the body of a patient, the catheter including an aspiration lumen in fluid communication with the distal end for removal of thrombus; positioning a distal end of the catheter in the region of a target thrombus; applying vacuum to the aspiration lumen; changing a valve state of a valve in fluid communication with the aspiration lumen to open or close the aspiration lumen; monitoring one or more pressures in the aspiration lumen; providing the valve state and the one or more monitored pressures to a trained machine learning model; and outputting a clot engagement state from the trained machine learning model.

A computer implemented method for a thrombectomy system is provided, comprising: receiving a valve state of a thrombectomy device that indicates valve open if a distal end of the thrombectomy device is in fluid communication with an aspiration source or valve closed if the distal end is not in fluid communication with the aspiration source; receiving one or more pressure measurements within the aspiration lumen; providing the valve state and the one or more pressures as inputs into a trained machine learning model; and outputting a clot engagement state with the machine learning model.

In one aspect, the method further includes indicating the output to a user.

In one aspect, the method further includes displaying the output on a display of the thrombectomy system.

In one aspect, the method further includes changing a mode of operation of the thrombectomy device in response to the output.

In one aspect, changing the mode of operation further comprises terminating aspiration.

In another aspect, changing the mode of operation further comprises initiating delivery of fluid streams at the distal end.

In one aspect, changing the mode of operation further comprises terminating delivery of fluid streams at the distal end.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIGS. 1A-1B show one example of a thrombectomy system.

FIGS. 2A-2B show one implementation of a clot detection waveform.

FIGS. 3A-3B show another implementation of a clot detection waveform.

FIG. 4 shows another implementation of a clot detection waveform.

FIG. 5 is yet another implementation of a clot detection waveform.

FIG. 6 is yet another implementation of a clot detection waveform.

FIGS. 7A-7B are representations of another thrombectomy system.

FIGS. 8A-8G show waveforms and analysis used by a clot detection algorithm to determine system and/or clot detection state of a thrombectomy system.

### DETAILED DESCRIPTION

Various aspects of this application relate to devices such as those disclosed in International Application No. PCT/US2021/020915, filed Mar. 4, 2021 (the ‘915 application), the disclosure of which is incorporated by reference herein for all purposes. The ‘915 application describes general mechanisms for capturing and removing clot. By example, the catheter may include a capture element such as an auger to break up and draw in a clot material into an aspiration lumen. In another example, multiple fluid streams are directed toward the clot to fragment the material.

The present technology is generally directed to thrombus removal systems and associated methods. A system configured in accordance with an embodiment of the present technology can include, for example, an elongated catheter having a distal portion configured to be positioned within a blood vessel of the patient, a proximal portion configured to be external to the patient, a fluid delivery mechanism configured to fragment the thrombus with pressurized fluid, an aspiration mechanism configured to aspirate the fragments of the thrombus, and one or more lumens extending at least partially from the proximal portion to the distal portion.

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section.

Additionally, the present technology can include other embodiments that are within the scope of the examples but are not described in detail with respect to the figures.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

Although some embodiments herein are described in terms of thrombus removal, it will be appreciated that the present technology can be used and/or modified to remove other types of emboli that may occlude a blood vessel, such as fat, tissue, or a foreign substance. Additionally, although some embodiments herein are described in the context of thrombus removal from a pulmonary artery (e.g., pulmonary embolectomy), the technology may be applied to removal of thrombi and/or emboli from other portions of the vasculature (e.g., in neurovascular, coronary, or peripheral locations). Moreover, although some embodiments are discussed in terms of maceration of a thrombus with a fluid, the present technology can be adapted for use with other techniques for breaking up a thrombus into smaller fragments or particles (e.g., aspiration only systems, ultrasonic, mechanical, enzymatic, etc.).

The headings provided herein are for convenience only and do not interpret the scope or meaning of the claimed present technology.

The present technology is generally directed to systems for removing unwanted material from a patient. Although described in terms of removal of thrombus, one will appreciate from the description herein that the systems and methods may be applied equally to other applications such as removal of benign growths, kidney stones, and more.

Aspiration-based systems typically remove blood while aspirating. Some have proposed blood-saving technologies, such as filters to “clean” the blood for re-insertion, but thus far they have found limited practical utility and come with additional complications.

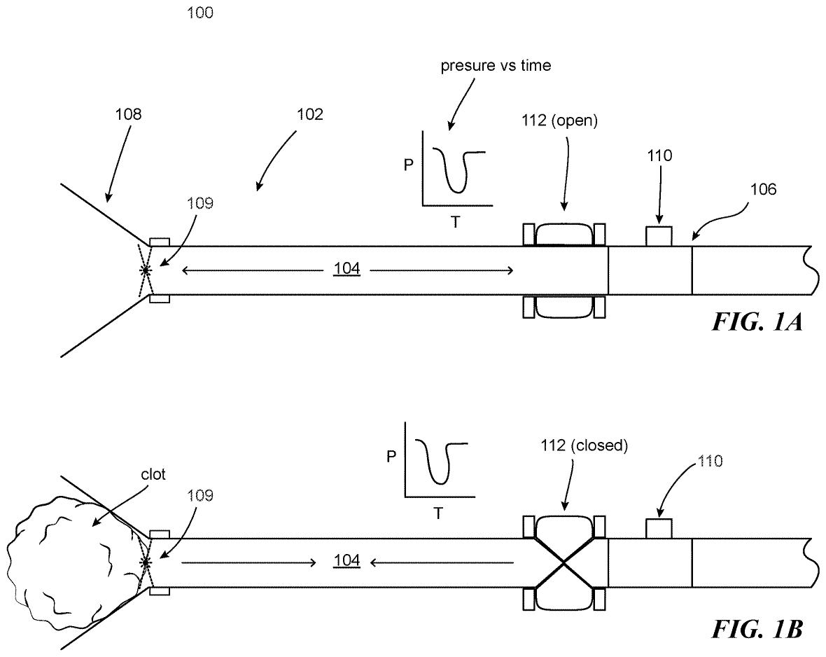

Turning to FIGS. 1A and 1B, various aspects of the present disclosure are directed to systems and methods for quickly detecting an operational state of a thrombectomy system 100 and/or improved control systems. The exemplary venous thrombectomy system 100 includes an elongated catheter 102 having a distal portion positionable within a blood vessel of the patient (e.g., an artery or vein), a proximal portion positionable outside the patient's body, an aspiration mechanism comprising an aspiration lumen 104 in the elongated catheter coupled to an aspiration source 106 configured to aspirate fragments of thrombus, and an optional fluid system configured to develop high velocity jets 109 to break up clot so it can be aspirated. The thrombectomy system can optionally include an expandable funnel 108 disposed at a distal end of the elongated catheter. In some embodiments, the systems herein are configured to engage a thrombus in a patient's blood vessel, break the thrombus into small fragments, and aspirate the fragments out of the patient's body. As used herein, “thrombus” and “embolism” are used somewhat interchangeably in various respects. It should be appreciated that while the description may refer to removal of “thrombus,” this should be understood to encompass removal of thrombus fragments and other emboli as provided herein.

In various embodiments, the system includes high velocity, intersecting fluid streams 109 at or near the funnel (e.g., saline jets) to break up thrombus so it can be passed through the aspiration lumen and removed through the low-profile catheter lumen. The high velocity jet streams may be configured for macerating, cutting, fragmenting, pulverizing and/or urging thrombus into the aspiration lumen and removed from a proximal portion of the catheter. The jet streams can also serve to allow fragments of the thrombus to be removed from the catheter even when the distal end/funnel is clogged or sealed with clot, and allows for maintenance of Large, hard clots would otherwise risk clogging inside the catheter such that the catheter must be removed from the patient, cleared, and re-introduced to the target site.

The aspiration lumen 104 may extend at least partially from the distal portion to the proximal portion of the catheter and in fluid communication with an aspiration source 106 (e.g., vacuum source). In various embodiments the aspiration source is positioned at the proximal end of the system and generates pressure which extends to the distal end of the aspiration lumen 104. The pressure or vacuum in the lumen causes the clot material to be ingested into the catheter and pulled through the aspiration lumen to a proximal end outside the patient's body.

In some embodiments, a pressure sensor 110 can be disposed within or in fluid communication with the aspiration lumen 104. The pressure sensor can be any conventional pressure sensor as known in the art. In one specific embodiment, the aspiration lumen itself can be configured as a fluid column, and the pressure sensor can be disposed proximal to the aspiration lumen (e.g., at or near the aspiration source). In the embodiment illustrated in FIGS. 1A-1B, the pressure sensor is shown proximal to the pinch valve 112. However, it should be understood that the pressure sensor can be disposed anywhere in the system that is in fluid communication with the aspiration lumen, including distal to the pinch valve, in the catheter 102 or funnel 108, in a handle of the device, or in the aspiration source. It should also be understood that multiple pressure sensors can be used in the system, in any of the locations described above or in fluid communication with the aspiration lumen. With this configuration, the pressure within the aspiration lumen can be accurately monitored with the pressure sensor 110.

The exemplary system includes one or more pinch valves 112 in communication with the aspiration pump. As would be understood by one of skill from the description herein, with the pinch valves closed (as shown in FIG. 1B) pressure will be decreased in the volume between the aspiration pump and valves. The pressure in the aspiration lumen, as measured by the pressure sensor 110, can thus be suddenly impacted by opening the pinch valves (as shown in FIG. 1A).

Typically systems rely on the aspiration pump to slowly build pressure in the system. In the exemplary embodiment, pressure is already built up and controlled precisely with the one or more pinch valves 112.

The systems described herein can include one or more processors or electronic controllers configured to control operation of the thrombus system. The one or more processors or electronic controllers can further be configured to execute instructions, algorithms, or computer implemented methods, e.g., algorithms for detecting or outputting a system state or clot detection/clot engagement state of the system. In some implementations, the system includes one or more processors and memory coupled to the one or more processors, the memory configured to store computer-program instructions, algorithms, software, firmware, or machine learning models/AI algorithms, that, when executed by the one or more processors, implement a computer-implemented method such as for detecting system or clot engagement state of a thrombectomy system.

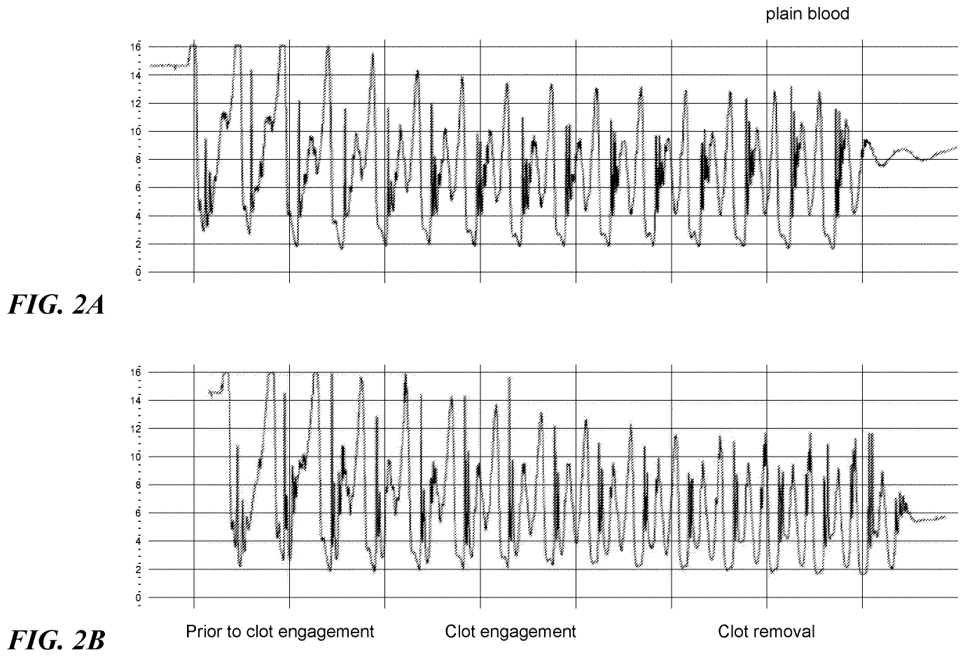

In the exemplary embodiment, pressure is cycled within the aspiration lumen by opening and closing the one or more pinch valves. FIGS. 2A-2B illustrate results for an exemplary system on that removes clot from a subject. FIG. 2A illustrates a pressure waveform when the thrombectomy system interacts only with blood, and FIG. 2B shows a pressure waveform when the thrombectomy system interacts with a clot and blood, including prior to engagement, when a clot is engaged, and after the clot has been removed with the thrombectomy system. As shown in FIG. 2B, the pressure in the aspiration lumen has not returned to the original, pre-engagement level (no full recovery) and takes longer to build vacuum with each cycle when the catheter has engaged clot.

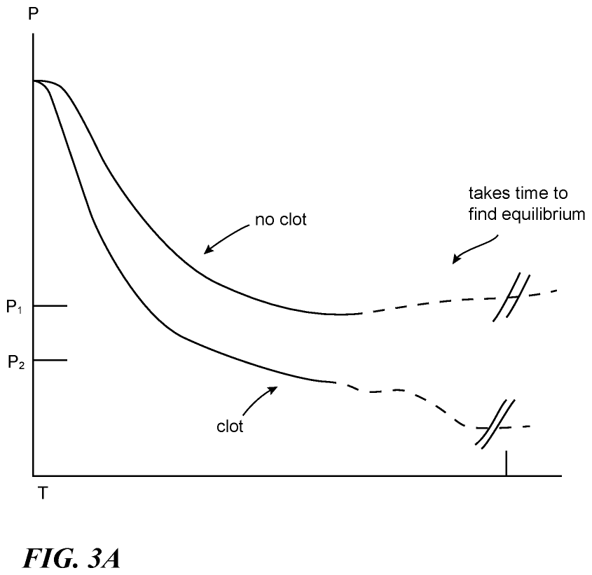

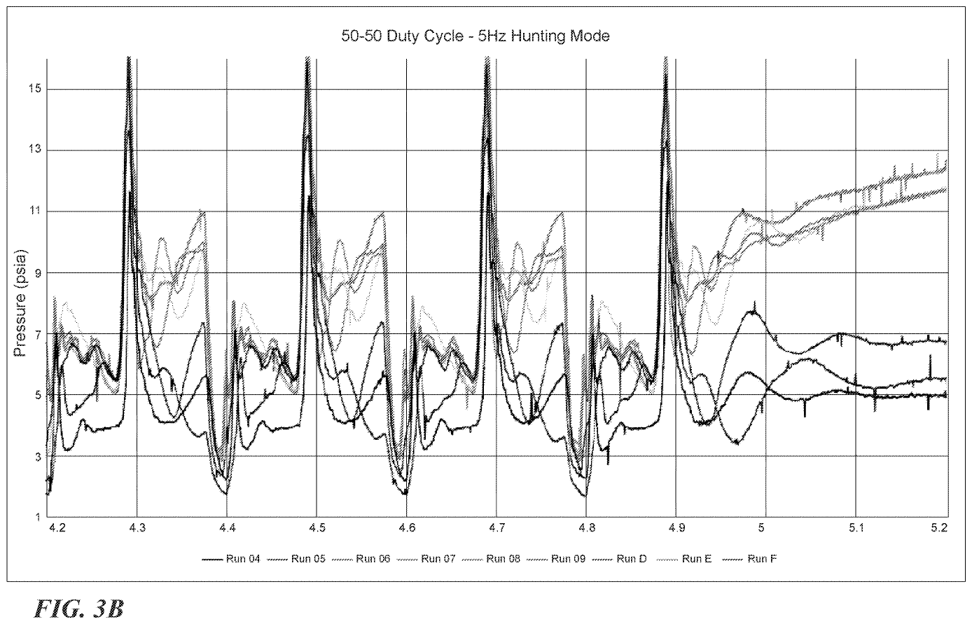

FIGS. 3A-3B illustrate pressure waveforms of the condition of an aspiration lumen pulling a vacuum with clot and without clot in a single cycle. In FIG. 3A, when no clot is present the aspiration lumen is open to the blood stream and will drop to a pressure level P1. When clot is engaged in the thrombectomy system (e.g., in the funnel or aspiration lumen), the aspiration lumen is partially or fully closed and the pressure will drop to a pressure level P2 that is lower than the pressure level P1, as shown. Over time, the pressure will continue dropping as the clot is engaged more. In both cases, however, it takes time (e.g. up to 20 seconds or more) to reach the maximum pressure and detect clot engagement. This explains why conventional systems which detect clot based on a pressure threshold take so much time to detect clot. The delay in detecting clot can be caused by, for example, compliance in the thrombectomy system itself, such as compliance in tubing connecting the aspiration lumen to the aspiration source, or compliance in the aspiration lumen, funnel, or other aspects of the system.

FIG. 3B shows pressure waveforms for a plurality of cases in which there is clot engaged with the funnel and where there is no clot engaged with the funnel. As shown in FIG. 3B, in cases where there is no clot engaged with the funnel, the pressure waveform rebounds back to a higher pressure when the pinch valve of the thrombectomy system is opened. When clot is engaged with the funnel, the pressure waveform remains relatively static or unchanged and does not rebound to the level seen when there is no clot engaged. For example, in one embodiment a clot detection algorithm can determine if a clot is engaged by evaluating the aspiration lumen pressure when the valve is opened and determining that a clot is engaged if the aspiration lumen pressure does not exceed a clot engagement threshold (e.g., a pressure level prior to engagement). As can be seen in the chart, the slope of the pressure waveform when there is no clot is significantly higher than the slope of the pressure waveform when clot is engaged, as there is relatively no pressure change when the pinch valve is opened, and clot is engaged. In one embodiment of a clot detection algorithm, the clot detection algorithm can determine if a clot is engaged by evaluating a slope of the aspiration lumen pressure when the valve is opened and determining that a clot is engaged if the aspiration lumen pressure does not exceed a clot engagement slope threshold. As described herein, the engaged clot serves to block or close off the aspiration lumen which prevents pressure rebound.

Referring again to FIGS. 2A-2B, a significant amount of ringing is observed such that the downslope in the aspiration lumen pressure measurement is not easily discernible. By contrast, the upslope has a more defined pattern. Recovery (in the exemplary test to 15 psi) is more consistent and presents a clear signal at the top of the waveform. In various embodiments, the system identifies clot (i.e. enters “clot engaged” state) based on these signature features. In various embodiments, the system identifies clot based on the way the peaks go down in each cycle. In various embodiments, the system will generate maximum vacuum even when it is not at maximum duty cycle (e.g., by building pressure while the clot blocks the aspiration lumen).

The exemplary system may include fluid jets and an aspiration lumen. In operation, before clot is engaged, the system will aspirate at least some of the jet fluid. Bench testing demonstrates that the system can detect capture of clot regardless of whether the fluid jets are on or not. However, in some embodiments even when the jets are off, fluid can still be pulled through the jet lumens into the aspiration lumen.

Compliance in the system would make it more challenging to identify clot engagement. However, bench testing demonstrated that the system can detect clot in spite of compliance in the system. Indeed, unlike conventional systems, in various respects the system in accordance with the inventions makes use of compliance in the system to detect clot.

A method of using the system to hunt for and detect clot engagement will now be described with reference to FIGS. 2-4. In use, the clinician already has located the general region of the target clot via imaging. The clinician positions the funnel of the thrombectomy catheter in the region of the target clot before turning on the system. Once in position, the system enters clot hunting mode. In this mode, the vacuum (aspiration) line (e.g., aspiration lumen 104) is open but is clamped off with a pinch valve (e.g., pinch valve 112) relatively rapidly. This reduces the amount of blood loss associated with vacuuming and hunting for thrombus and still provides a good pressure signature of when the system engages the clot. The challenge with conventional systems and algorithms is that it's hard to differentiate based on just vacuuming with the open vacuum sucking on the end of the device because there is so much resistance generated by the smaller diameter of the catheter as well as the hysteresis of the tubing.

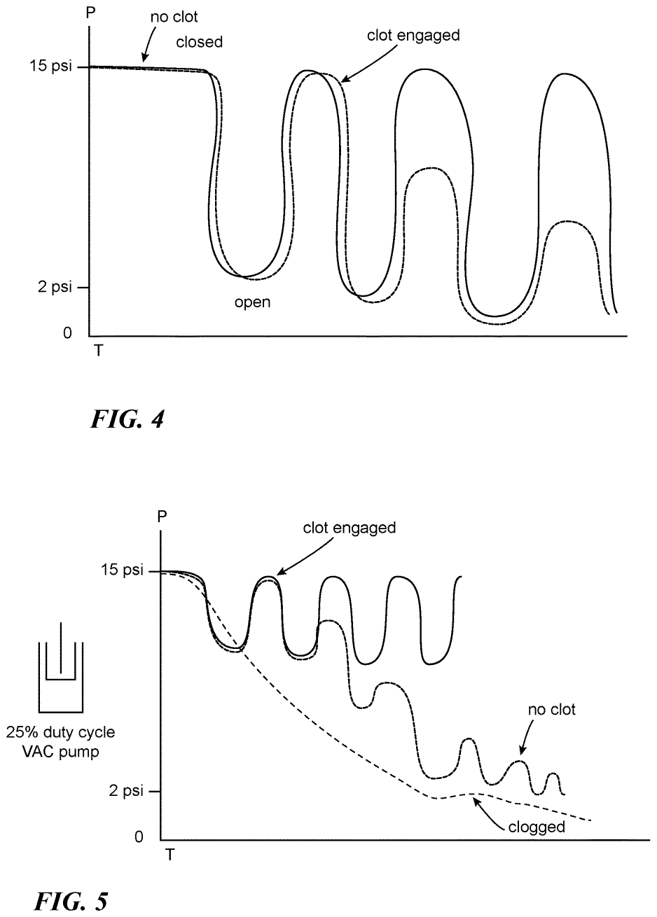

It has been found with the present system, however, that when the vacuum line is clamped off or closed and there is no clot present, the vacuum and the column of fluid is relieved. Referring to FIG. 4, two pressure waveforms are shown, a solid line pressure waveform showing pressure in the aspiration lumen when there is no clot engaged with the system, and a dashed line pressure waveform showing pressure in the aspiration lumen when clot is engaged. When cycling the pinch valve between open and closed, the pressure in the aspiration lumen rebounds back to atmospheric pressure when there is no clot present. By contrast, the pressure in the aspiration lumen does not return to atmospheric pressure (e.g., approx. 15 psi) when there is clot present. When the vacuum line is opened (e.g., by opening the pinch valve) the clot blocks the lumen and the pressure is not relieved by the anatomy. So the pressure in the aspiration lumen remains lower, and as the vacuum line is opened and closed the system generates more and more suction pressure on the clot. The pressure sensor (in fluid communication with the aspiration lumen) thereby detects engagement in the closed position based on whether or not the aspiration pressure rebounds to a threshold (e.g., atmospheric) pressure.

A specific implementation of the “clot hunting” algorithm is also described. First, the system can acquire a clot in the funnel with aspiration, as previously described. As mentioned above, the pressure in the aspiration lumen approaches vacuum when a clot is engaged in the funnel and aspiration is turned on. Next, the system can be configured to pinch off or close the aspiration lumen by closing the pinch valve located between the aspiration source and the pressure sensor. The system can initiate jetting and wait for the pressure waveform as monitored by the pressure sensor to rebound. If the pressure waveform stays down, it provides an indication to the user that there is still a clot present in the funnel of the device. However, if the pressure waveform goes back up, it provides an indication to the user that the clot has been cleared or partially cleared. This “clot hunting” process can be repeated throughout a clot removal procedure. In some embodiments, this process can be repeated every 3-5 seconds. The thrombus removal device of the present disclosure is configured to remove about 15 ml of material/blood/fluid per second. Therefore, by repeating this algorithm and process every 3-5 seconds, the system can, at a minimum, detect clot removal every 3-5 seconds while only aspirating between 45 ml of blood and 75 ml of blood in the event that the clot is removed. It should be understood that this process can be performed even more rapidly, potentially reducing clot removal detection time. The entire clot hunting algorithm can be performed in 300 ms or less. Detecting clot removal so quickly advantageously limits the amount of blood that is removed from the patient after clot removal.

FIG. 4 illustrates each of the two scenarios starting with the open lumen. The open scenario is similar to the solid line graph of FIG. 4 or a timestamp (single cycle) from FIGS. 2A-2B. When the system is clamped off without clot present the vacuum pressure quickly rebounds all the way back up to a threshold pressure such as atmospheric pressure+blood pressure (e.g., approx. 15 psi). The exemplary system can return to atmospheric pressure fairly quickly because of the pinch valve controls. In an exemplary embodiment, the system closes slowly (e.g. 100 milliseconds) and the system will easily rebound and go back up to the threshold pressure. In FIG. 4, the system may not be allowed enough time between cycles to allow for it to fully go back up but with more time it can fully rebound and stabilize with each cycle. This depends on various factors such as the resistance and compliance of the aspiration system. The system is then clamped again such that it can rebound back up to atmospheric pressure. If there is no clot present it will rebound and go back to 15 psi. If there is clot present it will not rebound and the vacuum pressure in the catheter remains because the clot is obstructing the end face of the system. The aspiration lumen is no longer open as a result of the engaged clot. In the exemplary graph the maximum pressure is 15 psi and the cycle time of the pinch valve is approximately 30 milliseconds. It should be understood that the threshold/maximum pressure and/or the cycle time of opening/closing the pinch valves can be adjusted.

Accordingly, the exemplary clot hunting algorithm is not relying on just absolute vacuum pressure. The algorithm is instead looking at the rebound pressure, rate, and/or waveform shape after the system clamps off the aspiration lumen. When the system is clamped off (e.g., aspiration lumen closed), the system evaluates whether (a) the pressure is alleviated and returns back to atmospheric or some other threshold pressure, or (b) the pressure largely remains the same or tapers off. If the latter-the pressure tapers off-the system identifies a blockage in the aspiration system which correlates to clot engagement. As shown in FIG. 4, the clot engaged line is less responsive and the system is never able to draw the full 15 psi vacuum again. Rather, the measured aspiration pressure tapers off which indicates clot is successfully engaged.

A method of detecting clot engagement in accordance with the present invention will now be described with reference to FIG. 4. The method includes generating a vacuum in the thrombectomy system by building pressure behind (proximal to) the pinch valve and then opening the pinch valve. Next the pinch valve is closed. The process is repeated for continuous cycles and the pressure in the aspiration lumen is monitored. The system monitors to see if the vacuum remains stable or more quickly returns to atmospheric. Based on the rate of pressure change (aka rebound) and/or pressure in the lumen with successive cycles the system detects clot engagement.

In various embodiments, the system turns off aspiration if clot engagement is not detected within a predetermined period or number of duty cycles of the aspiration source. The step of turning off aspiration can serve to prevent or reduce the amount of blood removed from the subject by the thrombectomy system.

The system and method described herein has been found to be able to accurately detect clot engagement in a matter of seconds or less. In general, the system can detect clot engagement with just a few duty cycles. By comparison, conventional systems and algorithms simply aspirate until pressure in the aspiration lumen drops below a threshold which can take significant time and put the patient at risk. In use, such conventional systems take 20 seconds or more and the results can be indeterminate.

In various embodiments, the system uses the above technique in conjunction with other parameters. In various embodiments, the method includes use of a pressure threshold.

In various embodiments, the system coordinates control of the jets and aspiration. In various embodiments, the system will transition to activate the jets when clot is successfully engaged. In this manner aspiration time is limited and then system can quickly remove clot once it has been found. In various embodiments, the system makes use of advanced techniques like machine learning to identify parameters, pressure thresholds, and the like to improve the system controls.

The system may be designed to operate at a lower vacuum level than a target pressure at the working end of the catheter. The graphs above can be shifted up and the amount of vacuum required is reduced if the system generates the target clot engagement signature when closed at a lower vacuum pressure. So the vacuum pump can be configured to operate at a lower speed/pressure, but the system can still discern the “clot engagement” signature pattern. When the system clamps off, the vacuum still remains on the clot. The vacuum generated on the clot can be brought down to a lower level, e.g. 2 psi, because the aspiration lumen is blocked. A higher level of vacuum can be created when hunting for the algorithm and the pressure is reduced when clot is captured. The system can automatically reduce pressure or a signal can be generated to given an indication to the clinician. The reduced vacuum pressure in the engaged mode reduces the amount of blood loss.

FIG. 5 illustrates the pressure in the aspiration lumen over several cycles using the system and methods described above. As shown, the vacuum pump is controlled to generate the same pressure profile, but instead of pulling full vacuum, the system is configured to pull reduced vacuum (e.g. half or quarter duty cycle). The vacuum pump is then stopped and the pinch valve is opened. If the catheter is engaged with clot, the lumen is clogged and the vacuum begins to build up in the catheter even though the vacuum pump is not running at full duty cycle. In general, the system is configured so the vacuum pump generates vacuum pressure in small quantities and periods instead of running at high pressure all at once. Put another way, the system pulls small amounts of vacuum over the course of one or a few large cycles. The system can still generate sufficient vacuum when the catheter is engaged with a clot because the pressure ratchets up more and more on the clot and the actual pressure in the lumen goes down despite the fact that the system is not applying a lot of absolute pressure. In this case it can be seen that the whole step function shifts downward in pressure towards absolute vacuum and weakens when the catheter is engaged with clot. The result is the system removes even less blood because the pump is running at lower vacuum. Moreover, the pump requirements are reduced because it does not need to generate high vacuum pressures.

A benefit of the exemplary valved system is to alleviate the challenges of capacitance of the fluid canister from the operation. The collection canister has an air volume, which in turn has a capacitance. It thus normally takes time to reach absolute vacuum in the system. The exemplary pinch valves, however, circumvent this challenge by enabling the vacuum pump and canister to get to equilibrium and then the pinch valves can be opened. The exemplary system with the valves allows for a vacuum hammer effect to generate higher vacuum even with smaller pump load. This also enables instantaneous or near instantaneous vacuum at the desired level. A conventional system might have a 60 cc (60 mL) syringe and the user must draw the full vacuum. The exemplary system provides the same and/or higher level of vacuum with the addition of more control and less demands on the components.

The valved configuration also enables quicker and improved control over pressure. By comparison to conventional systems, the user does not need to wait for capacitance in the system to be overcome. The clinician can precisely and quickly control pressure with the valves. As understood from the description above, the more responsive control of pressure also enables the system to more quickly detect clot engagement based on pressure recovery. The system does not need extra time to wait for capacitance in the system.

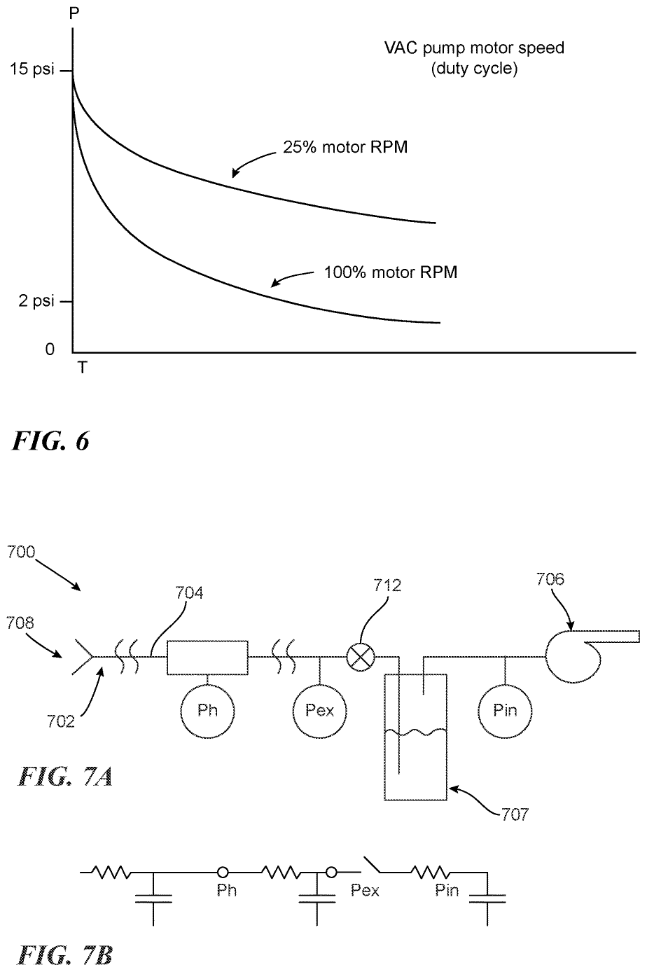

With reference to FIG. 6, the system enables “digital control” with precise, responsible control of pressure at any desired location in the fluid/pressure system. This digital control enables the vacuum pump to be throttled and reduce the duty cycle, power demands, and pump RPM. It has been found from bench tests that reducing the vacuum pressure by half or even 75% still provides sufficient pressure to engage and detect clot. For example, in some embodiments the pump may be configured to generate 7 psi and 10 psi, respectively. By controlling the pinch valve to clamp and release the aspiration lumen, when engaged with clot can still pull vacuum down on the clot. The pressure sensor and controls can then identify pressure going down similar to FIG. 5 with less blood/fluid removal.

FIGS. 7A-7B are representations of another embodiment of a thrombectomy system 700 that can include some or all of the features previously described, including elongated catheter 702 having a distal portion positionable within a blood vessel of the patient (e.g., an artery or vein), a proximal portion positionable outside the patient's body, an aspiration mechanism comprising an aspiration lumen 704 in the elongated catheter coupled to an aspiration source 706 configured to aspirate fragments of thrombus into an aspiration cannister 707, and an optional fluid system configured to develop high velocity jets to break up clot so it can be aspirated (not shown). The thrombectomy system can optionally include an expandable funnel 708 disposed at a distal end of the elongated catheter. A pinch valve 712 can be configured to selectively open or close the aspiration lumen, as previously described. In some embodiments, the systems herein are configured to engage a thrombus in a patient's blood vessel, break the thrombus into small fragments, and aspirate the fragments out of the patient's body.

In the embodiment of FIG. 7A, the system can include more than one pressure sensor configured to measure a pressure within the aspiration lumen or the aspiration source, including a pressure sensor Pₕ disposed within our fluidly coupled to the aspiration lumen (e.g., at the handle of the device), pressure sensor Pₑₓ disposed within or fluidly coupled to the aspiration lumen at the pinch valve, and pressure sensor Pᵢₙ disposed within or fluidly coupled to the aspiration source.

FIG. 7B is a representation of thrombectomy system 700 that shows resistance and capacitance in the system corresponding to the locations of the pressure sensors Pₕ, Pₑₓ, and Pᵢₙ.

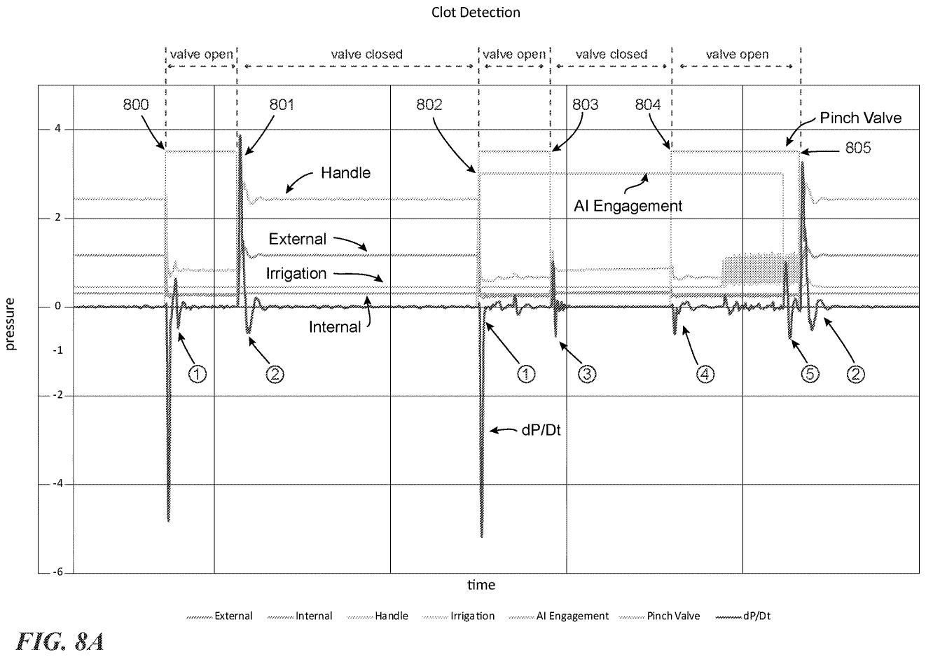

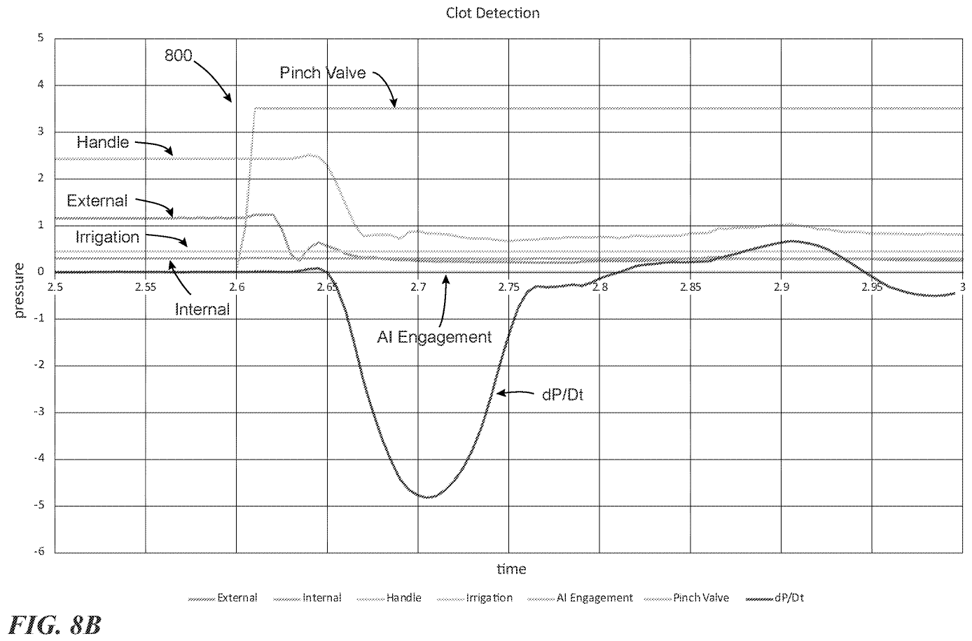

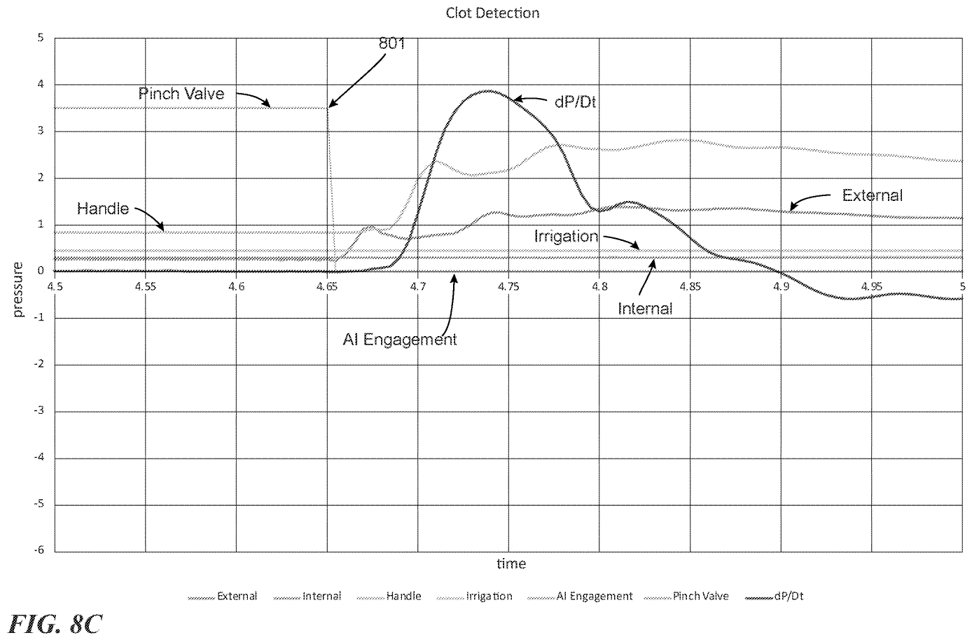

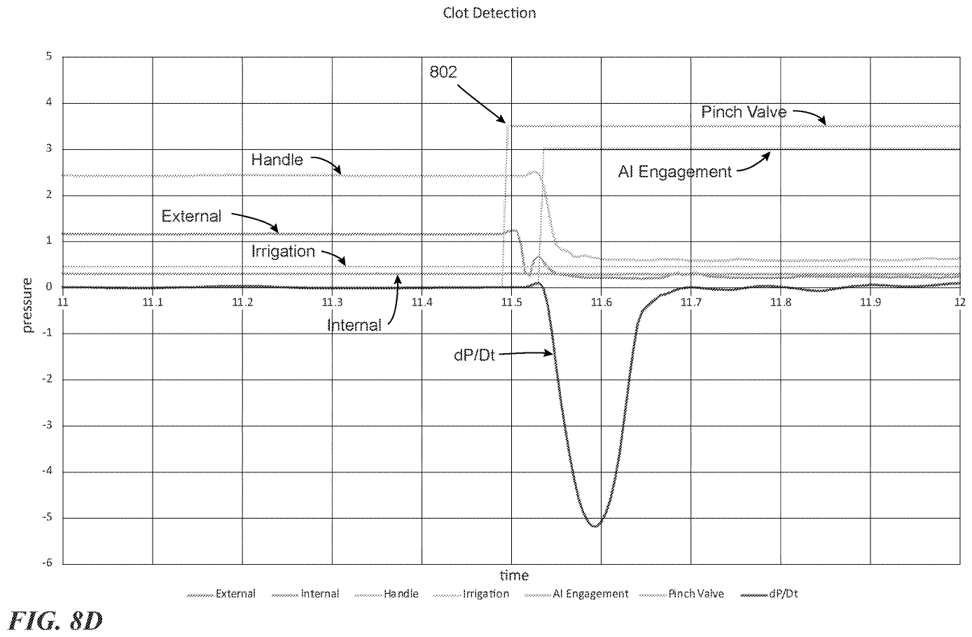

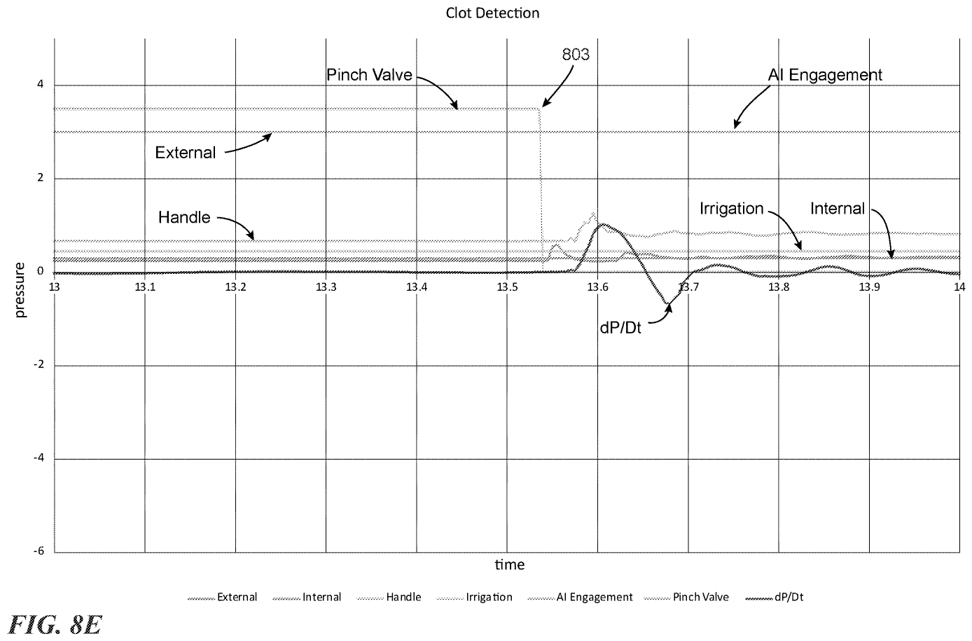

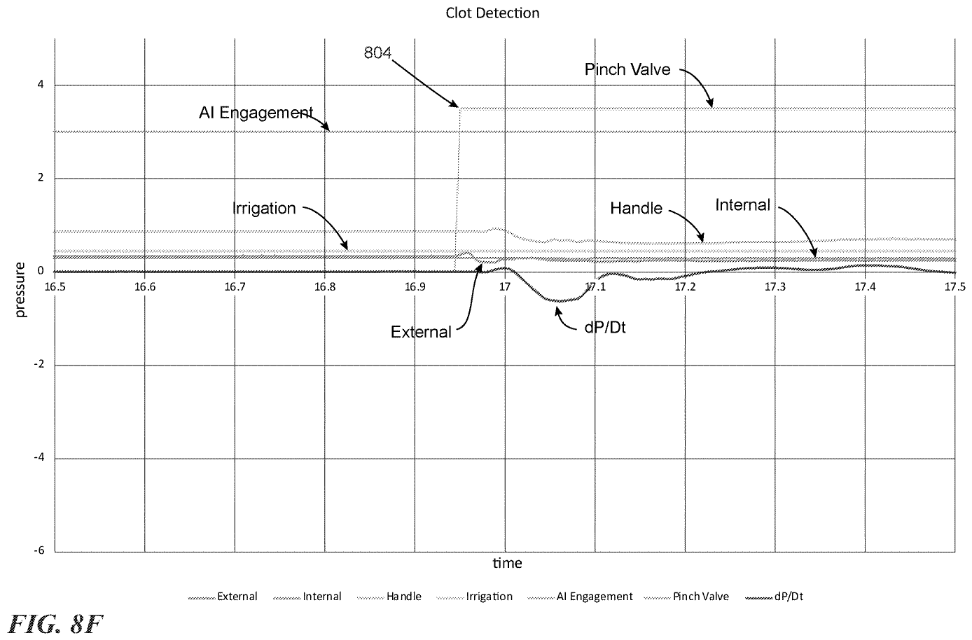

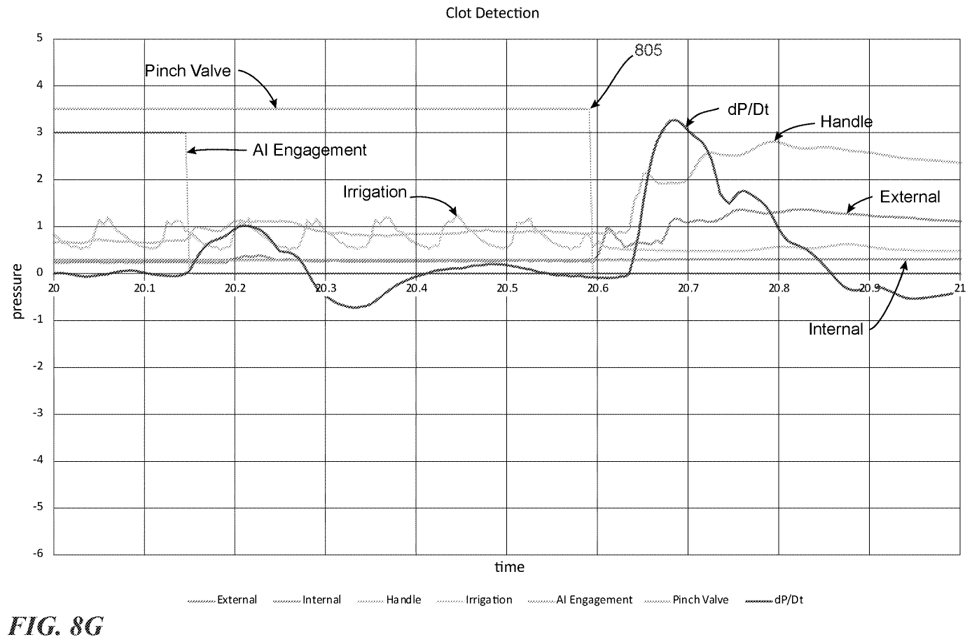

FIGS. 8A-8G show additional charts of pressure waveforms used for clot detection using the system of FIG. 7. In FIGS. 8A-8G, the clot detection algorithm can be a traditional algorithm or can alternatively be an artificial intelligence algorithm or alternatively an output from a trained machine learning model. FIG. 8A shows pressure waveforms for each of the pressure sensors described in FIG. 7A, including a pressure waveform corresponding to Pₕ, Pₑₓ, and Pᵢₙ. An irrigation pressure (e.g., a jetting or fluid stream pressure) is also shown in FIG. 8A. The chart of FIG. 8A shows various system states corresponding to the opening and closing of the pinch valve and resulting measured pressures Pₕ, Pₑₓ, and Pᵢₙ. For example, system state 1 in FIG. 8A represents the pinch valve opening with no clot engaged with the thrombectomy device. System state 2 in FIG. 8A represents the pinch valve closing with no clot engaged with the thrombectomy device. System state 3 represents the pinch valve closing with clot engaged with the thrombectomy device. System state 4 in FIG. 8A represents the pinch valve opening with no clot engaged with the thrombectomy device. Finally, system state 5 represents the clot aspirated from the thrombectomy device.

In a first embodiment, a traditional algorithm can be used to evaluate or assess the various pressure waveforms to determine if clot is engaged or not engaged with the thrombectomy system. In one embodiment, the algorithm can assess the change in pressure waveforms over time (dP/dt) to determine if clot is engaged or not. The dP/dt waveform is shown in FIG. 8A. In some examples, the dP/dt waveform is calculated as a linear fit filter over a sequential set of points (e.g., points collected over a set period of time such as 10 msec, 20 msec, 30 msec, 40 msec, 50 msec, or longer etc.). However, other filters can be used such as median filters, linear filters, Fourier filters, etc. In one implementation, the valve can be cycled between on/off after priming the system to set/normalize dP/dt for variations in compliance between individual catheters. Comparing system state 1 to system state 3, it can be seen that the dP/dt waveform has a large negative peak at system state 1 (valve opening with no clot) compared to system state 3 (valve closing with clot engaged). Furthermore, there is low frequency ringing in system states 1 (valve opening with no clot) and 2 (valve closing with no clot) compared to a large positive peak in states 3 (valve closing with clot engaged) and 4 (valve opening with clot engaged). These pressure waveform behaviors and parameters can be used by the algorithm to determine if clot is engaged or not.

In some embodiments, the initial opening and closing of the pinch valve when a clot is not engaged can be used to calibrate or normalize the pressure readings of the thrombectomy device to an initial state. Further opening and closing of the valve can then be compared to this initial state to determine clot engagement, no clot engagement, clogging, and/or clot clearing.

For example, referring to FIG. 8A, at time reference 800, the valve is opened (e.g., transitions from a closed state to an open state) and no clot is detected, resulting in a large peak negative response with a low frequency. This large peak negative can be a baseline peak negative and the low frequency can be a baseline low frequency against which future peak negative and frequency responses are compared against to determine system state. Similarly, at time reference 801, the valve is closed (e.g., transitions from an open state to a closed state) and no clot is detected, resulting in a large peak positive response with a low frequency. This large peak positive can be a baseline peak positive and the low frequency can be a baseline low frequency against which future peak positive and frequency responses are compared against to determine system state.

For example, in one implementation, a clot detection algorithm will identify a large negative peak (e.g., a negative pressure larger than a clot detection threshold, or alternatively, similar to or within an accepted ratio of the baseline peak negative, such as up to 50%/75%/85%/95% of the baseline peak negative) and will also identify a low frequency response similar to the baseline low frequency response in a dP/dt waveform when the valve is opening to determine that a clot is not engaged with the system (system state 1). Alternatively, a clot detection algorithm will identify a peak positive that is higher than a clot detection threshold, or below a ratio of the baseline peak positive and will also identify a high frequency ringing in a dP/dt waveform that is a higher frequency than the baseline frequency when the valve is opening to determine that a clot is engaged with the system (system state 4).

In another implementation, a clot detection algorithm will identify a large positive peak (e.g., a positive pressure larger than a clot detection threshold or similar to the baseline positive peak) and or a low frequency response similar to the baseline frequency response in a dP/dt waveform when the valve is closing to determine that a clot is not engaged with the system (system state 2). Alternatively, a clot detection algorithm will identify a positive peak below a clot detection threshold and/or a high frequency response higher than the baseline frequency response in a dP/dt waveform when the valve is closing to determine that a clot is engaged with the system (system state 3).

The clot detection algorithm can determine that a clot has been aspirated by identifying a spike in a dP/dt waveform (positive or negative) while the valve is in the open state (system state 5). This can also be referred to as detecting that the clot has been cleared.

In another embodiment, a real-time clot clear detection algorithm can use the pressure signals to determine when the system has cleared or removed a clot that was previously engaged. The algorithm can employ a parametric approach that uses a correlation function to an averaged “clot relief signature” to detect that a clot has been cleared. In one embodiment, pressure signals can be filtered through a low pass filter to clean the signals, and then the algorithm can take the log of the vacuum signals, take the difference, and normalize. The algorithm can then run a correlation against a pre-defined profile. Sensitivity can be controlled with correlation threshold and sampling window size, with improving accuracy of detection as window size increases. Another parametric approach can pass the vacuum signals through a low pass filter, double-differentiate the signals, apply a blanking period around the valve open and valve close states, and then apply a threshold detector. In yet another parametric approach, the algorithm can low pass filter the external vacuum signals, double-differentiate the signals, apply a blanking period around the valve open and valve close (the valve motion gives large artifacts in this signal), and then applied a threshold detector to determine engagement or clot clearing.

Still referring to FIG. 8A, in a second embodiment, the thrombectomy system can employ a trained machine learning model to determine system state (e.g., clot engaged, no clot engaged, clot aspirated). In some implementations, the machine learning model(s) can be trained by tagging a system state while pressure measurements are obtained during a procedure or with a training data set. The training can include an input of valve state (e.g., open/closed, transitioning from open to closed, transitioning from closed to open), clot engagement status (not engaged, engaged), pressure values from any of the pressure sensors, and other system parameters (e.g., jetting on/off, aspiration on/off, etc.). In one example, the training has three inputs: 1) pinch valve state, 2) aspiration source and/or aspiration cannister pressure, and 3) pinch valve pressure. Optionally, the pressure reading from the handle can be added as an input during training for increased accuracy. For example, a user can tag a system state of no clot engaged, clot partially engaged, or clot engaged/clogged during a procedure or with a training data set, and the machine learning model can be trained to correlate data with a given system state. The trained model can then be used as described above to determine system state and/or clot engagement during a procedure. The machine learning model can provide an output indicative of the system state and/or the clot state. For example, the machine learning model can output that the system is not engaged with a clot, is engaged with a clot, is clogged, or has cleared a clot. The machine learning model can further output a probability of clot engagement (e.g., 25%/50%/75%/100% probability of clot engagement). Alternatively, the machine learning model can give a binary output of clot engagement (e.g., 0=no clot engaged, 1=clot engaged).

In FIG. 8A, at time reference 800, the pinch valve opens resulting in a drop in pressure measurements recorded by Pₕ and Pₑₓ, while Pᵢₙ remains relatively stable. A large peak negative in the dP/dt waveform is shown, indicating that initially when the valve is opened there is no clot engaged. The AI model does not detect engagement at this time, so the “Engagement” line that represents the AI model remains at a value of 0. FIG. 8B is a close up view of the waveforms occurring after time reference 800. This corresponds with system state 1.

Back to FIG. 8A, at time reference 801, the pinch valve closes resulting in an increase in pressure measurements recorded by Pₕ and Pₑₓ, while Pᵢₙ remains relatively stable. A peak positive in the dP/dt waveform is shown, indicating that initially when the valve is opened there is no clot engaged. The AI model does not detect engagement at this time, so the “Engagement” line that represents the AI model remains at a value of 0. FIG. 8C is a close up view of the waveforms occurring after time reference 800. This corresponds with system state 2.

Referring still to FIG. 8A, the AI model is shown identifying a clot engaged shortly after the valve is opened as identified by the AI engagement line indicated with time reference 802. FIG. 8D is a close-up view of this time period following time reference 802. In FIG. 8D, it can be seen that shortly after the valve is opened, both Pₕ and Pₑₓ begin to drop, while Pᵢₙ remains relatively stable. A large peak negative in the dP/dt waveform is shown, indicating that initially when the valve is opened there is no clot engaged. However, the AI model detects engagement shortly thereafter as shown in FIG. 8D. Since the clot is not yet engaged when the valve is opening, this is associated with system state 1, but it can be seen that engagement is detected by the AI model shortly thereafter.

FIG. 8A shows time reference 803, in which the valve closes and clot engagement is still detected by the AI model. This corresponds with system state 3. FIG. 8E is a close-up view of the time period that follows time reference 803. It can be seen that pressure measurements recorded by Pₕ and Pₑₓ do not rebound as high as they did in FIG. 8C when no clot was engaged, while Pᵢₙ remains relatively stable. This is an indication that a clot is engaged since the clot is blocking the distal end of the aspiration lumen preventing pressure rebound.

FIG. 8F shows the time period around time reference 804, when the valve re-opens and clot is still engaged with the device. The AI model continues to show clot engagement. All the sensed pressures remain relatively stable. This corresponds with system state 4.

FIG. 8G shows the period around time reference 805 in FIG. 8A, with the valve closing shortly after the AI model identifies that the clot is no longer engaged with the thrombectomy device. In some examples, this can be associated with a system state that indicates that the clot is cleared. With the various pressure sensors (particularly Pₕ), the system can sense a transitory signal. In one example, the system can look at vacuum relief curves (e.g., a time constant of pressure restoration and/or flow rates) to determine that the clot that was previously engaged with the system has been cleared. In FIG. 8G, it can be seen that shortly after the AI model determines clot is no longer engaged, there is an increase or peak in the dP/dt waveform and both Pₕ and Pₑₓ. There is a further rebound or spike of the Pₕ and Pₑₓ after the valve closes while Pᵢₙ remains relatively stable.

While described in terms of pinch valves, one of skill will appreciate from the description herein that other elements may be used to achieve the described clamp effect and pressure control.

1. A system for removing thrombus, the system comprising:
an elongated catheter having at least one aspiration lumen configured to remove thrombus material;
an aspiration mechanism fluidly coupled to the aspiration lumen and configured to reduce pressure in the aspiration lumen;
a pressure sensor configured to monitor a pressure inside the aspiration lumen;
a valve disposed between the pressure sensor and the aspiration mechanism; and
an electronic controller operatively coupled to the pressure sensor and the valve, the electronic controller being configured to open and close the valve and monitor the pressure within the aspiration lumen to determine if a clot is engaged with the elongated catheter or if the clot has been at least partially removed.

2. The system of claim 1, wherein the electronic controller is configured to close the valve every 3-5 seconds of therapy.

3. The system of claim 2, wherein a cycle time to open or close the valve is approximately 300 ms or less.

4. The system of claim 1, wherein the electronic controller is configured to determine that a clot is engaged with the elongated catheter when a monitored pressure does not substantially increase after closing the valve.

5. The system of claim 1, wherein the electronic controller is configured to determine that a clot has been at least partially removed when a monitored pressure increases after closing the valve.

6. The system of claim 1, wherein the system is configured to remove no more than 45 ml of blood from the patient before detecting that the clot has been at least partially removed.

7. The system of claim 1, wherein the valve has a valve state that comprises an open state, a closed state, an opening state, or a closing state.

8. The system of claim 7, wherein the electronic controller is configured to determine that a clot is engaged if the valve moves from a closed state to an open state and the pressure within the aspiration lumen fails to exceed a clot engagement threshold.

9. The system of claim 7, wherein the electronic controller is configured to determine that a clot is engaged if the valve moves from a closed state to an open state and a slope of the pressure within the aspiration lumen fails to exceed a clot engagement threshold.

10. The system of claim 1, wherein the electronic controller is configured to assess a change in pressure within the aspiration lumen over time to determine if clot is engaged with the elongated catheter or if the clot has been at least partially removed.

11. The system of claim 10, wherein the change in pressure within the aspiration lumen over time is an input into a correlation function against a pre-defined clot detection profile.

12. The system of claim 10, wherein the change in pressure within the aspiration lumen over time are filtered and normalized.

13. The system of claim 7, wherein the electronic controller is configured to provide the pressure within the aspiration lumen and the valve state to a trained machine learning model to determine if clot is engaged with the elongated catheter or if the clot has been at least partially removed.

14. A system for removing thrombus, the system comprising:
an elongated catheter having at least one aspiration lumen configured to remove thrombus material;
an aspiration mechanism fluidly coupled to the aspiration lumen and configured to reduce pressure in the aspiration lumen;
a valve coupled to the aspiration mechanism, the valve having a valve state that comprises an open state or a closed state;
a first pressure sensor disposed distal to the valve and configured to monitor a first pressure inside the aspiration lumen;
a second pressure sensor disposed proximal to the valve and configured to monitor a second pressure inside the aspiration lumen; and
an electronic controller operatively coupled to the pressure sensor and the valve, the electronic controller being configured to open and close the valve and monitor the first pressure and second pressure within the aspiration lumen to determine if a clot is engaged with the elongated catheter or if the clot has been at least partially removed.

15. The system of claim 14, wherein the electronic controller is configured to close the valve at most every 3-5 seconds of therapy.

16. The system of claim 15, wherein a cycle time to open or close the valve is approximately 300 ms or less.

17. The system of claim 14, wherein the electronic controller is configured to determine that a clot is engaged with the elongated catheter when a monitored pressure does not substantially increase after closing the valve.

18. The system of claim 14, wherein the electronic controller is configured to determine that the clot has been at least partially removed from the elongated catheter when a monitored pressure increases after closing the valve.

19. The system of claim 14, wherein the system is configured to remove no more than 45ml of blood from the patient from at least partially removing the clot to detecting that the clot has been at least partially removed.

20. The system of claim 14, wherein the electronic controller is configured to determine that a clot is engaged if the valve moves from a closed state to an open state and the first and second pressure within the aspiration lumen fails to exceed a clot engagement threshold.

21. The system of claim 14, wherein the electronic controller is configured to determine that a clot is engaged if the valve moves from a closed state to an open state and a slope of the first and second pressure within the aspiration lumen fails to exceed a clot engagement threshold.

22. The system of claim 14, wherein the electronic controller is configured to assess a change in pressures within the aspiration lumen over time to determine if clot is engaged with the elongated catheter or if the clot has been at least partially removed.

23. The system of claim 22, wherein the change in pressures within the aspiration lumen over time is an input into a correlation function against a pre-defined clot detection profile.

24. The system of claim 22, wherein the change in pressures within the aspiration lumen over time are filtered and normalized.

25. The system of claim 14, wherein the electronic controller is configured to provide the first and second pressures within the aspiration lumen and the valve state to a trained machine learning model to determine if clot is engaged with the elongated catheter or if the clot has been at least partially removed.

26-55. (canceled)
